(19) United States
(12) Patent Application Publication
(10) Pub. No.: US 2019/0275284 A1
(43) Pub. Date: Sep. 12, 2019

(54) PALM DEVICE AND METHOD THEREOF FOR MAINTAINING MIND CONCENTRATION

(71) Applicants: Michael SAPIR, BEER-SHEVA (IL); Anatoly RIVIN, BEER-SHEVA (IL); Ruslan SEVRUK, BEER-SHEVA (IL)

(72) Inventors: Michael SAPIR, BEER-SHEVA (IL); Anatoly RIVIN, BEER-SHEVA (IL); Ruslan SEVRUK, BEER-SHEVA (IL)

(21) Appl. No.: 16/281,140

(22) Filed: Feb. 21, 2019

Related U.S. Application Data
(60) Provisional application No. 62/638,981, filed on Mar. 6, 2018.

(51) Int. Cl. A61M 21/00

(52) U.S. Cl. CPC A61M 21/00; A61M 2021/0022; A61M 2205/502; A61M 2205/3569; A61M 2210/083; A61M 2205/8206; A61M 2209/088

(57) ABSTRACT

A method and a device for maintaining mind concentration, is presented here. This device is intended for ADD children and students, and for people that are apt to fall asleep unintentionally while doing their task. The device is mainly composed of a microcontroller, batteries and few switches. The microcontroller provides a digital timer, and it generates a sequence of sets of signals at the end of a given time interval. Those are sent via a wire to the micro motor which is housed in a wrist belt that is situated on the user hand. As the motor is actuated it vibrates and shakes gently the user hand; Those stop daydreams, and irrelevant thoughts, if any, while they do not disturb attention or concentration, on behalf, of the special compact structure.

### BACKGROUND and PRIOR ART

The current invention relates to addressing concentration disorders and specifically to a palm device and method thereof for maintaining mind concentration.

In the specification and claims which follow hereinbelow, the term “student” is intended to mean anyone being taught frontally and/or studying by himself or in a classroom setting. A “student” includes but is not limited to: children and young adults. Many students are diagnosed as having insufficient attention abilities. The students' ability to concentrate is weak. Such a disability is may be termed Attention Deficit Disorder (ADD) or Attention-Deficit/Hyperactivity Disorder (ADHD). The inattention is frequently caused by a noise inside or outside a classroom, or even by a word said by the teacher/lecturer or by others in the classroom. ADD or ADHD are typified by being easily impressed by noisy events and other events. Those trigger an arousal of various associations, which lead to irrelevant thoughts or even daydreams. As a result, the student may lose connection with the lecture or with the student's task.

In the specification and claims which follow hereinbelow, the term “concentration disorder” is intended to mean ADD, ADHD and/or any other similar attention and/or concentration disorder, as well as any other condition adversely effecting concentration, such as, but not limited to fatigue and/or lack of sleep. Similarly, the expression “maintaining mind concentration” is intended to mean a method or actions directed to deal with a concentration disorder and to thus maintain concentration.

One approach for coping with this concentration disorders, is by using chemicals and drugs, such as, but not limited to: Ritalin© (methylphenidate HCl). The drug serves to stimulate the central nervous system, thereby helping the user to stay focused. However, there are side-effects such as: sleeping disorders; headaches; a degree of addiction; and nervousness, among others. Therefore, the use of Ritalin© and similar stimulants is highly debated in the professional literature. Moreover, in many countries, a prescription by a doctor is needed for its use. As a result, a non-drug solution to concentration disorders is preferable. The prior art includes a number of non-drug solutions, as noted further hereinbelow.

Another approach uses mental exercises; for example, the ATENGO (4) method. According an example of the method, an ADD patient exercises quick identification of various objects. This approach has some success in improving mental/concentrating capabilities. However, the method involves many hours of exercises, and it takes a significant time to achieve a substantial improvement. As a result, some consider this approach as insufficient. There is therefore, a need for a friendly device and method that help the ADD student right-away.

While surveying the prior art, a group of devices that were intended for a tired driver, are also of interest, since, they are basically connected to the problem of reduced concentration. However, as they were directed to a driver, they are not adequate solutions to the ADD problem, as is noted hereinbelow.

In U.S. Pat. No. 4,361,834, dated 1978, whose disclosure is incorporated by reference, King Johnnie. describes a device directed to help a tired driver. The inventor suggests an electronic circuit, comprising, an alarm device, and batteries that are housed in wristband, and connected electrically to a (pressure) switch which is supported by a hand glove. A firm contact-pressure between the hand and a contact surface (steering wheel), signifies a normal situation, while, a relaxed pressure signifies a tired driver, and the alarm is sounded. Obviously, the pressure criterion for detecting a dozy user, and the sound alarm are not appropriate for enhancing attention of an ADD student.

Love, in U.S. Pat. No. 5,012,226 dated 1991, whose disclosure is incorporated by reference, describes a device also directed to a tired driver, including user interaction with a timer of an electronic circuit. At the end of a predetermined period an alarm is sounded. However, if within the period the driver presses a switch, the timer restarts, and the actuation of the buzzer-alarm at that period is deterred. This patent too, is not appropriate for stopping daydreams and enhancing attention because of the sound alarm.

Many other publications include descriptions of devices using similar techniques: a timer that activates an alarm device that is housed in a wristband; a switch that controls deactivation, and is placed on a glove, a finger; etc.

In U.S. Pat. No. 5,684,462, whose disclosure is incorporated by reference, Gold describes a device (again) directed to help a tired driver, the device composed of a box containing an electrical circuit. He also uses a digital timer, to actuate an alarm which is visual or audio. And on the box is placed a special switch, that controls the deactivation option for the alert device

Power, in US Patent Application Publication no. 20060250256, whose disclosure is incorporated by reference, describes a device also directed to a tired driver, suggesting a glove or straps on the fingers to mount there few pressure-sensors, while the alarm device is housed in wristband. Clearly, while the driver holds normally the steering wheel, grip-pressure is developed and applied to the pressure sensors. Consequently, the alarm does not sound. But as the driver falls asleep his grip relaxes, and the sensors immediately activate the alarm.

In US Patent Application Publication no. 20090189772, whose disclosure is incorporated by reference, Christi et al. describe a device (again) directed to help a tired driver and/or a non-driving fatigued user, based on the interaction between user input and a microcontroller to decide immediately whether the user is fully alert, or tends to fall asleep, whereby an alarm is activated. This solution involves few sensors, which could cause quite significant inconvenience to the user. Therefore, its effectiveness regarding attention disorders, is doubtful. Consequently, a satisfactory solution that is adequate for the ADD problem is needed.

Now, by combining few of the electronic techniques used in the prior art mentioned above—with the understanding that various states of inattention are the outcome of daydreams or of irrelevant thoughts, an effective solution to the ADD problem, emerges.

This is presented here.

SUMMARY OF INVENTION

An approach and a device for coping with the ADD problem, are presented here. The approach is based on the understanding that the inattention state, is the outcome of irrelevant thoughts, or of short daydreams. In order to stop them the device generates, vibrations that gently shake the “dreamer” until he becomes aware of his real surroundings. The device fits the following demands:

1) The alarms should cut off daydreams or irrelevant thoughts that occupy the user's mind.

2) The alarms should not disturb the user, so long as he is attentive or concentrated.

According the first demand: Alarms are applied each 17 seconds for 10 seconds. The values 17 sec, and 10 sec, are only exemplary values. These parameters have been chosen as compromise, between short enough time for surveying inattention, and not too frequent alarms, that might be a nuisance. They were tested along many sessions of use.

To fulfill the second demand: The components of the device were assembled into a compact configuration, that enables to operate the device without any movement of the hand nor of even the eyes; Consequently, the use of the device does not involve significant mind activity, and therefore, the user can stay attentive or concentrative.

Not the least, is the potential of the device, to provide alertness to those who want, or need to stay awake, while they are bored or tired.

### LIST OF FIGURES

The invention is herein described, by way of example only, with reference to the accompanying drawings, wherein:

FIGS. 1, 2A, and 2B, are pictorial representations of a palm device system for maintaining concentration, in accordance with embodiments of the current invention;

FIG. 3 is a block diagram of the palm device system of FIGS. 1, 2A, and 2B, in accordance with embodiments of the current invention; and

FIGS. 4A and 4B, are a flow chart showing an operation sequence of the palm device system shown in previous figures, in accordance with embodiments of the current invention.

### DETAILED DESCRIPTION

Embodiments of the current invention relate to addressing concentration disorders and specifically to a palm device and method thereof for maintaining mind concentration.

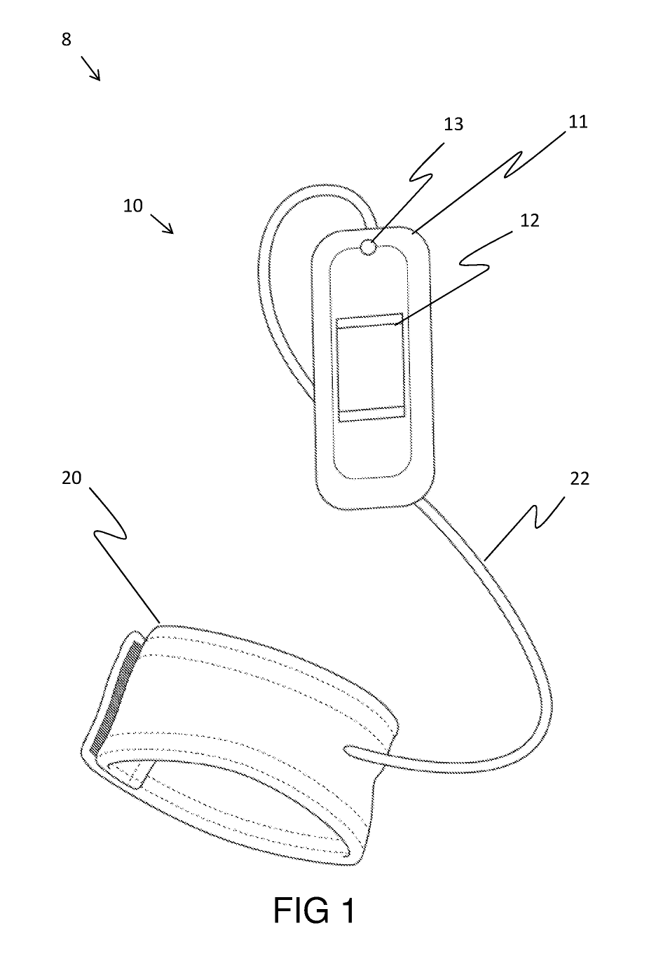

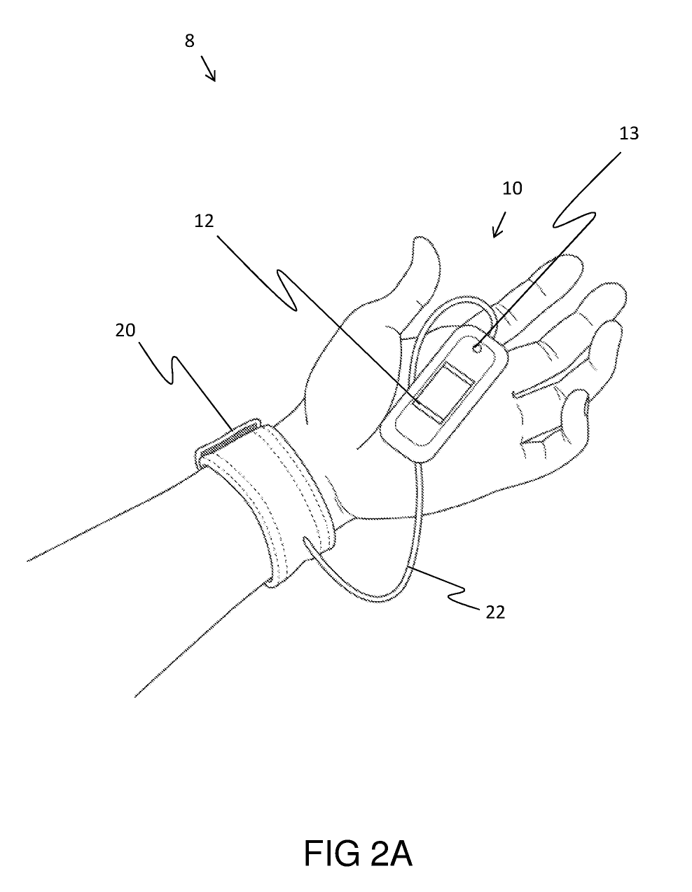

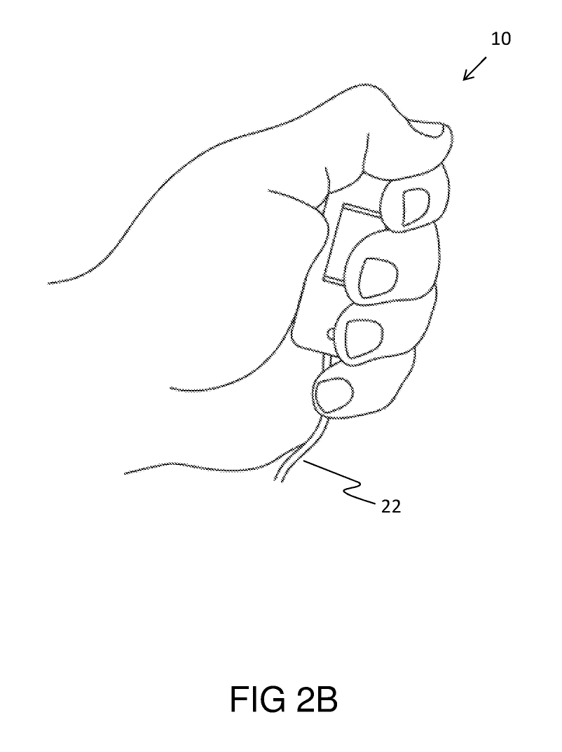

Reference is currently made to FIGS. 1, 2A, and 2B, which are pictorial representations of a palm device system for maintaining concentration 8, in accordance with embodiments of the current invention. Palm device system 8 includes: a palm device 10; and a wrist-worn vibration unit 20. The palm device further includes: a case 11; a command/control switch 12; and a LED indicator light 13. Exemplary signal cord 22 serves to connect the wrist-worn vibration unit with the palm device. Electronic components, as described further hereinbelow, in addition to the command/control switch and the LED indicator light, are housed in case 11.

As shown in FIGS. 2A and 2B, palm device 10 is held in the palm of a user while the user's fingers may rest naturally on command/control switch 12. This switch, serves to receive and control user-interface commands, such as, but not limited to: on/off; stop vibrations; restart a new sequence of signal-sets; and change-time interval—all as described further hereinbelow. To increase comfort and reliability two equivalent knobs are available at the ends of the rocker switch

Although command/control switch 12 is represented in the figures as having an exemplary rocker switch configuration, embodiments of the current invention include any similar switch configuration, such as, but not limited to: a button switch; and a contact membrane switch.

The LED indicator 13 light serves to indicate various operational modes of the device, as further described hereinbelow.

A nonconcentric micro motor 38 is configured in wrist-worn vibration unit 20, the micro motor serving as a small vibration source, as known in the art, and as further described hereinbelow. The wrist-worn unit is configured to be worn like a wrist watch, as shown in FIG. 2A. Although an exemplary Velcro®-like closure is shown in FIG. 2A, the wrist-worn unit may be secured with any typical closure known in the art. Various functionalities of palm device system 8 and of palm device 10 are described hereinbelow.

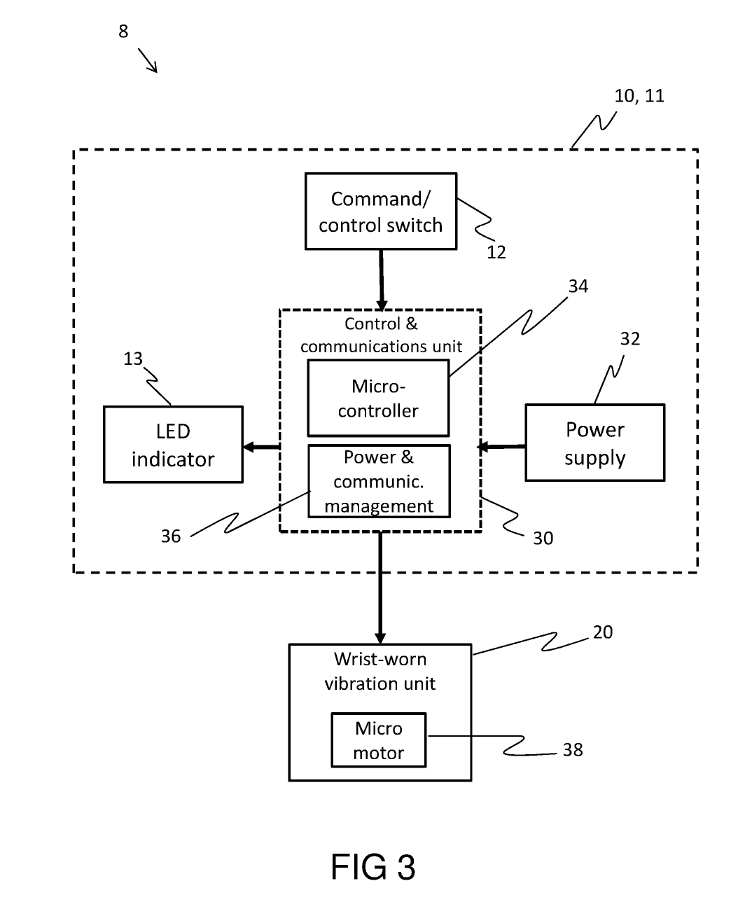

Reference is currently made to FIG. 3, which is a block diagram of palm device system 8 of FIGS. 1, 2A, and 2B, in accordance with embodiments of the current invention. Palm device system 8 is identical in notation, configuration, and functionality to that shown in the previous figures and elements indicated by the same reference numerals and/or letters are generally identical in configuration, operation, and functionality as described hereinabove. Palm device 10 includes: a case 11, which houses: a control and communication unit 30; a power supply 32; command/control switch 12 and LED indicator light 13 as noted hereinabove. Control and communication unit 30 serves to command and to control wrist-worn unit 20, as further described hereinbelow.

Control and communication unit 30 includes: a microcontroller 34, which controls the wrist-worn unit and interfaces and receives commands from command/control switch 12; and a power and communications management unit 36, which serves to receive power from power supply 32 and to provide power to the microcontroller, the LED indicator, and to the wrist-worn unit. Signal communications and power are transferred by control and communication unit 30 to wrist-worn unit 20—via the signal cord 22.

Although signal cord 22 is shown in FIGS. 1, 2A, and 2B, embodiments of the current invention optionally or alternatively include wireless control and communication of the wrist-worn unit by palm device 10, in which case signal cord 22 is not present.

Power supply 32 includes two batteries, that are separated to-begin-with by an insulating strip.

The micro-controller unit 34, generates a sequence of sets of alarm signals. The sets are separated by a predetermined time interval of (say), 17 sec, and include, (say), 10 signals that appear at the end of each time interval, during (say) 10 sec. Those are sent to the micro motor 38, causing it to vibrate as each signal arrives. The vibrations stimulate the user's wrist/hand and rouse a reflexive desire to stop them. This might be accomplished easily, by pressing lightly either end of the rocking switch. This pressing act stops the current vibration—set and restarts a new sequence of the 17 sec—sets. Besides, though most important, the activity of the vibrations on the user hand, and the action of pressing the switch-each of these actions, interrupts daydreams and serves to stop them.

Thus, the user becomes aware again of the “real situation”—for example, the teacher's lecture or the textbook he is reading.

The choice of 17 seconds for the time interval between vibrations has been found to be effective and comfortable for use of the device over an extended period, such as hours. From one standpoint, the system functions to void/disrupt long daydream periods. From another standpoint, a shorter time interval could lead to a nuisance of too-frequent vibrations. Nonetheless, the time interval may be changed, as described hereinbelow.

It is important to ensure that use of the device does not by itself disturb an attentive or concentrating user. Wherefore this, the device configuration components are enclosed in a compact and comfortable configuration (FIG. 2A). The case is typically held comfortably in the user's palm, with one or more fingers resting on the command/control switch (FIG. 2B). Thereby the user can press the switch quickly for stopping the vibrations, without having the need to look for, or to stretch his hand to hold it. This simple act of pressing the switch scarcely involves mental attention and is generally a reflexive act. This situation is like that of a driver reacting to the traffic conditions by signaling or using the brake pedal—while maintaining full concentration of the traffic. So, like the driver, a concentrating user is not disturbed by using the device. Moreover, following a short training period, the act of pressing the switch in reaction to wrist vibration becomes, an automatic reflex, just like driving. As a result, an attentive user can stop the vibrations, and at the same time, maintain mind concentration

Micro motor 38 was chosen as an alarm/alert device because it vibrates quietly and thus, it can be used also in a classroom and in an auditorium. For the alarm vibrations to be effective, the motor is configured in the wrist-worn unit so that vibrations are felt on the user's wrist.

### Operation Procedures

The operation procedures that are illustrated in the flowchart hereinbelow, can be summarized as follows:

First, a “cycle” is defined as follows: “The LED is blinking for 17 seconds, followed by vibrations that last 10 seconds, while the LED still blinks”.
1) Powering up the device, while using for the first time: The insulating strip that separates the batteries, is pulled away. Then the LED turns on, giving steady light for 20 seconds. Afterwards, as the LED starts blinking—the device is ready, and the cycles are going on.
2) Vibrations: After the LED blinks for 17 seconds the motor vibrates for 10 seconds. Then a new cycle starts
3) Stopping vibrations. Just press lightly one end of the rocker switch. Vibrations stop and then a new cycle starts.
4) Shut down the device: Press continuously the knob of the switch until blinking stops (around 4 seconds), release the knob, then press it once again for a short while. Then the device is sleeping.
5) Activating the (sleepy) device: Press continuously the knob (For about 3 seconds) until the LED blinks. A cycle starts.
6) Reducing the number of vibrations: Press the knob before the end of the 17 seconds interval. A new cycle starts.

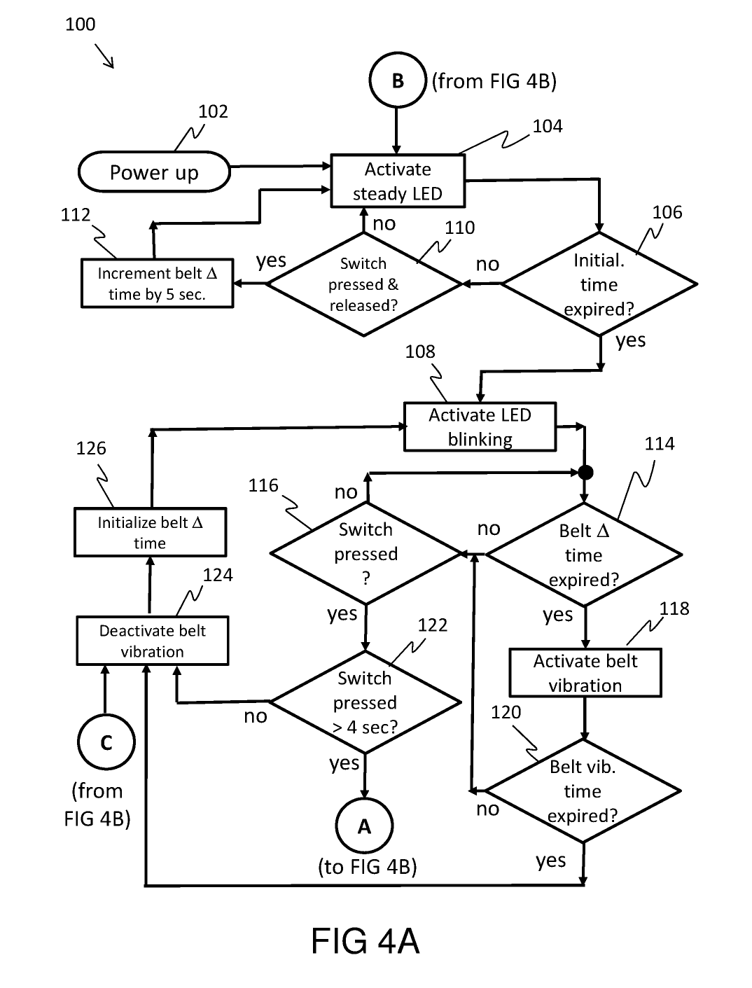

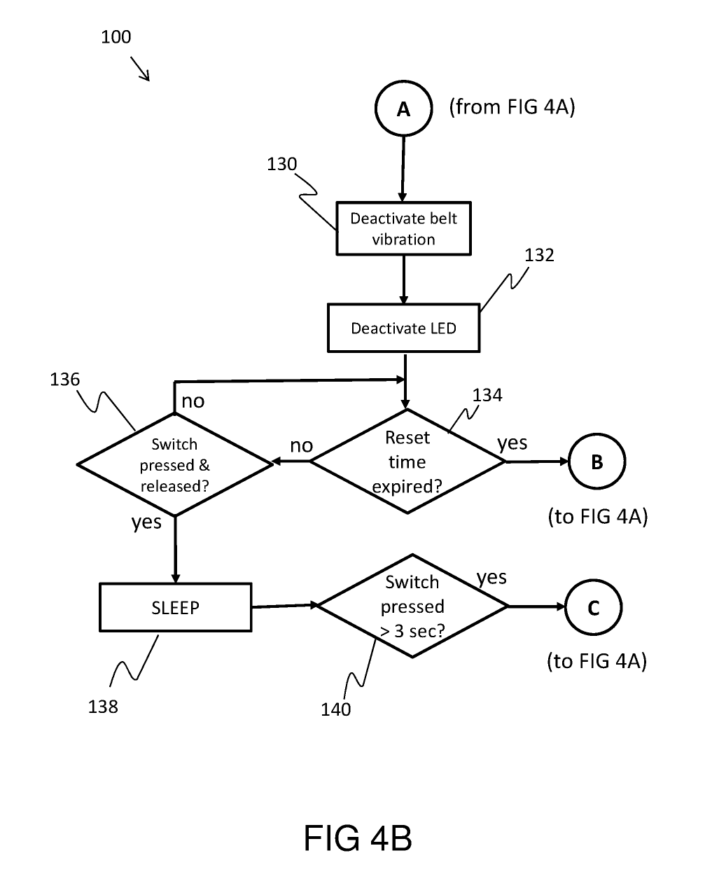

Reference is currently made to FIGS. 4A and 4B, which are a flow chart 100 showing an operation sequence/procedure of the palm device system shown in the previous figures, in accordance with embodiments of the current invention.

In the following description the term “LED” is intended to mean “LED indicator light”, as shown/described, inter alia, in FIGS. 1 and 2A. In the following description, the term “belt” is intended to mean “wrist-worn vibration unit” and the term “switch” is intended to mean “command/control switch”—all as shown/described, inter alia, in FIGS. 1 and 2A hereinabove.

A detailed explanation of the flowchart is given as follows:

Step 102 (Power up) is an initial step to apply power to the system, such as after batteries are in position and the insulating strip is removed. This applies to first use of the device; (However, if the device has already been in use, i.e. the insulating strip has already been removed then the procedure starts at the SLEEP step-point—138 in FIG. 4B and will be discussed later). In step 104 (“activate Steady LED”), the LED is illuminated continuously (“steady”) for 20 sec, At step 106, it is checked if “initial time (20 sec) is expired”. If “yes”, then continue to step 108, where a cycle is started: e.g. the LED starts blinking and the time interval (17 sec)) begins to count down. In step 114 The interval time (17 sec) is checked to determine if it is expired. If the answer is “no”, command is transferred to step 116. There it is checked whether “Switch was pressed” If “no”, then transfer to 114 again.

If at 114 the belt interval time (17 sec) is expired, then go to 118 (“Activate belt vibration”) and the belt vibration sequence commences. Then in step 120, it is checked: “is vibration time (10 sec) expired?”. If “yes”, vibrations are stopped at step 124, interval time is initialized at 126, and a new cycle starts at 108.

Now back to step 120: If vibration time did not expire, transfer is done to step 116. There if, the switch was not pressed, then the flow is transferred to step 114, 118, and 120. Now back to step 116: If the switch there was pressed, and not for more the 4 seconds then transfer takes place to 124 and 126 and a new cycle starts at 114.

Back to 122: If there the switch was pressed for more then 4 sec, then transfer is done to step A (In FIG. 4B). At 130, vibrations are stopped if they exist, and at step 132 the LED is deactivated. At 134, (Reset time expired?), where the reset time (10 sec) is the time elapsed after LED deactivation. If yes, a transfer is done to step B—at FIG. 4A, and then to 104, where the LED gives steady light for 20 seconds, and afterwards a new cycle starts.

Back to step 134 in FIG. 4B: If reset time did not expired then transfer is done to step 136, where (“Switch is pressed & released?”) If “no” return to 134″). If “yes” transfer to step 138 (SLEEP). Then at step 140, if the switch is pressed for more than 3 sec, go to step C at FIG-4A, and start a new cycle at step 124.

Finally, back to step 106. If initial time did not expire, e.g. the LED is giving steady light, transfer is done to step 110, (“Switch pressed & released?”), if “no” go to point 106. If “yes”, increase time interval by 5 seconds at point 112 and go to 106

Now we refer to the situation where the device had already been in use. Then the flowchart starts at step 138 in FIG. 4B.

### Changing Time Intervals

The time interval can be changed by the following steps: Starting from a state where the LED blinks, the user should press continually the knob for about 4 seconds until the LED is going off, and then release. Now wait until the LED is lighting steadily, (about 10 seconds). This state, of continuous lighting continues for 20 seconds. During those 20 seconds each pressing and releasing of the knob, increases the time interval as follows: pressing once and releasing, or avoid pressing, sets it to be 17 seconds, twice pressing and releasing, sets it to be (17+5) seconds, three times of pressing and releasing sets it to be (17+5+5) seconds, and so forth. Each pressing, and releasing, should be somewhat elongated (say 1 second), and is followed by stopping the continuous light for a short while. After those (say 20) seconds, the LED blinks, and the device enters a new cycle, with a new value for the time interval.

### Prevention of Unwilling Sleep

The device can be helpful for keeping a fatigued user awake—and thus maintain mind concentration. The device is used the same manner as described hereinabove. The device serves to provide vibrations to the user's wrist. The vibrations serve to disturb the napping stage from developing into full sleep. In this way the device can be useful to enable people to continue with their task, even though they would normally be starting to fall asleep.

It will be appreciated that the above descriptions are intended only to serve as examples, and that many other embodiments are possible within the scope of the present invention as defined in the appended claims.

### Some Generalization

The time intervals mentioned above where chosen specifically for the preferred embodiment. But other time intervals might do as well. The same holds for the numerical values of the other mentioned parameters. The picture of the device is not obligatory; Other geometries might do as well. Also, the connection between the case and the motor might be via wireless means.

For some other circumstances, another embodiment may be of advantage, where the vibrating motor is substituted by a buzzer.

And finally, the pushbutton switches may be replaced by touch sensors.

### REFERENCES

1) https://www.cbsnews.com/news/adhd-meds-may-cause-sleep-problems-in-kids/
2) https://ritalinsideeffects.wordpress.com/2012/01/26/terrible-headaches-on-ritalin/ https://www.additudemag.com/ritalin-and-headaches/
3) https://www.dependency.net/learn/ritalin/ https://www.dependency.net/learn/ritalin/
4) http://www.attengo.co.il/

PATENT CITATIONS

| Cited patent | | Filing date | Publication date | Applicant | Title |
|---|---|---|---|---|---|
| 5) | U.S. Pat. No. 4,361,834 | Dec. 13, 1978 | Nov. 30, 1982 | King Johnnie L | Safety alarm device |
| 6) | U.S. Pat. No. 5,012,226 | Feb. 23, 1990 | Apr. 30, 1991 | LOVE; Samuel D | Safety alertness - - |
| | | | | | monitoring system |
| 7) | U.S. Pat. No. 5,684,462 A | Jan. 16, 1996 | Nov. 4, 1997 | Bert Joseph Gold | Driver attention alarm |
| 8) | US20060250256A1 | Mar. 28, 2006 | Nov. 9, 2006 | Power Jerome | Sleep alert device. |
| 9) | US20090189772A1 | Jan. 28, 2009 | Jul. 30, 2009 | Christ Randolph et al, | Stay-alert device |

1. A palm device system for maintaining mind concentration of a user, the system comprising:
a palm device, including: a case; a control and communications unit; a command/control switch; and two batteries; and
a wrist-worn vibration unit, configured, to be worn on the wrist and connected to the palm device, the wrist-worn unit housing a micro-motor;
wherein the palm device system is adapted to controllably operate the micro motor to activate vibrations to the wrist, thereby interrupting and serving to stop daydreamers, while subsequent operating the command/control switch stops the vibrations.

2.-12. (canceled)

13. The palm device system of claim 1, wherein the control and communications unit further comprises: a microcontroller configured to control the wrist-worn vibration unit and to interface and receive commands from the command/control switch; a power and communications management unit configured to receive power from the batteries and to provide power to the microcontroller, and to communicate with the wrist-worn unit.

14. The palm device system of claim 13, wherein the wrist-worn vibration unit is connected to the palm device by at least one chose from the list including: a signal cord and; a wireless connection.

15. The palm device system of claim 13, wherein the palm device is configured to be positioned in the user's palm, with one or more fingers resting on the command-control switch.

16. The palm device system of claim 15, wherein the command-control switch is configured to be operated quickly to stop the vibrations, without the need for the user to look for, or to stretch his hand to hold the switch.

17. The palm device system of claim 13, wherein the ability to stop the vibrations quickly, enables the attentive or the concentrating user to stay undisturbed.

18. A method of operating a palm device system for maintaining mind concentration of a user, the method comprising the steps of:
a. positioning a palm device, including: a case; a control and communications unit; a command/control switch; and two batteries, in a user's palm;
b. wearing a wrist-worn vibration unit and connecting the wrist-worn unit to the paint device, the wrist-worn unit housing a micro-motor;
c. configuring a microcontroller to control the wrist-worn vibration unit and to interface and receive commands from the command/control switch;
d. activating the microcontroller to command the micro motor to activate vibrations to the user, thereby interrupt and serve to stop daydreams.
